(19) United States
(12) Patent Application Publication
(10) Pub. No.: US 2013/0317108 A1
(43) Pub. Date: Nov. 28, 2013

(54) DERMATOLOGICAL FOAMS OBTAINED FROM A GEL OR SUSPENSION CONTAINING A COMBINATION OF ADAPALENE AND BENZOYL PEROXIDE

(71) Applicant: Emmanuelle At, Antibes (FR)

(72) Inventor: Emmanuelle At, Antibes (FR)

(73) Assignee: GALDERMA RESEARCH & DEVELOPMENT, Biot (FR)

(21) Appl. No.: 13/997,125

(22) PCT Filed: Dec. 22, 2011
(86) PCT No.: PCT/FR2011/053158

§ 371 Date: Aug. 16, 2013

(30) Foreign Application Priority Data
Dec. 23, 2010 (FR) 1061165

(51) Int. Cl. A61K 9/12; A61K 31/327; A61K 31/192

(52) U.S. Cl. CPC A61K 9/122; A61K 31/192; A61K 31/327
USPC 514/569

(57) ABSTRACT

Intermediate compositions, and in particular gels and suspensions for foam compositions, are described that include adapalene and benzoyl peroxide in combination. Also described; is the dermatological use of such compositions.

The present invention relates to foam compositions based on a combination of adapalene and benzoyl peroxide, in particular as topical dermatological compositions, especially for treating dermatoses such as acne.

The use of several classes of active principle is a therapeutic tool to which recourse is frequently made, especially for the treatment of dermatological disorders.

Specifically, various antifungal agents such as allylamine derivatives, triazoles, antibacterial or antimicrobial agents, for instance antibiotics, quinolones and imidazoles, are conventionally combined in the treatment of dermatological diseases. It is also known practice to use peroxides, D vitamins and retinoids for the topical treatment of various pathologies related to the skin or mucous membranes, in particular acne.

The combination of several local treatments (antibiotics, retinoids, peroxides, zinc) is also used in dermatology for increasing the efficacy of active principles and for reducing their toxicity (Cunliffe W. J., J. Dermatol. Treat., 2000, 11 (suppl. 2), S13-S14).

The multiple application of different dermatological products may be quite burdensome and constraining for the patient.

The advantage of seeking to obtain a novel treatment that is effective on dermatological complaints in a stable composition affording good cosmeticity, enabling a single targeted application, and also a use that is pleasant for the patient, may thus be appreciated.

Among this panoply of therapeutic agents proposed to those skilled in the art, there was nothing to encourage said person to combine, in the same composition, benzoyl peroxide and a retinoid.

Specifically, formulating such a composition poses several problems.

Firstly, the efficacy of benzoyl peroxide is associated with its decomposition when it is placed in contact with the skin. Thus, it is the oxidizing properties of the free radicals produced during this decomposition which lead to the desired effect. Thus, in order to maintain optimal efficacy for benzoyl peroxide, it is important to prevent its decomposition before use, i.e. during storage.

However, benzoyl peroxide is an unstable chemical compound, this instability making it difficult to be formulated in finished products.

The solubility and stability of benzoyl peroxide were studied by Chellquist et al. in ethanol, propylene glycol and various mixtures of polyethylene glycol 400 (PEG 400) and water (Chellquist E. M. and Gorman W. G., Pharm. Res., 1992, vol. 9: 1341-1346).

Benzoyl peroxide is particularly soluble in PEG 400 and ethanol, as shown in the following table:

| | Solubility of benzoyl |
|---|---|
| Solvent | peroxide (mg/g) |
| PEG 400 | 39.6 |
| Ethanol | 17.9 |
| Propylene glycol | 2.95 |
| Propylene glycol/water | 0.36 |
| mixture (75/25) | |
| Glycerol | 0.15 |
| Water | 0.000155 |

Said document moreover states that the stability of benzoyl peroxide is greatly influenced by the chemical composition of the formulation and by the storage temperature. Benzoyl peroxide is extremely reactive and degrades in solution at low temperature on account of the instability of its peroxide bond.

The authors thus find that benzoyl peroxide in solution degrades more or less quickly in all the solvents studied as a function of the type of solvent and of its concentration.

The degradation times (half-life time) of benzoyl peroxide in PEG 400 (0.5 mg/g), in ethanol and in propylene glycol are, respectively, 1.4, 29 and 53 days at 40° C.

Such degradation does not enable the preparation of a product intended for sale.

It is moreover known that benzoyl peroxide is more stable in water and propylene glycol when it is in suspension (i.e. in dispersed form), since it is not degraded after 90 days of storage in these solvents.

Thus, to limit the problem of rapid instability of benzoyl peroxide in solution, it proved to be advantageous to formulate benzoyl peroxide in dispersed form. However, this type of formulation is not entirely satisfactory insofar as degradation of benzoyl peroxide in the finished product is still observed.

Another difficulty to overcome for the preparation of a composition comprising both benzoyl peroxide and a retinoid is that the majority of the retinoids are particularly sensitive to natural oxidation, visible light and ultraviolet light, and, as benzoyl peroxide is a strong oxidizing agent, the chemical compatibility of these compounds in the same formulation poses numerous physical and chemical stability problems.

A stability study over 24 hours only on two retinoids was performed by combining two commercial products, one containing a retinoid (tretinoin or adapalene) and the second based on benzoyl peroxide (B. Martin et al., Br. J. Dermatol. (1998) 139 (suppl. 52), 8-11).

The presence of the formulation based on benzoyl peroxide brings about a very rapid degradation of the oxidation-sensitive retinoids: it is measured that 50% of the tretinoin is degraded within 2 hours, and 95% within 24 hours. In the composition in which the retinoid is adapalene, no degradation of the adapalene was measured within 24 hours. This study confirms that benzoyl peroxide is degraded and degrades the oxidation-sensitive retinoids over time by gradually releasing benzoic acid into finished products.

Now, it is clear that the degradation of benzoyl peroxide and of retinoids is undesirable insofar as it is detrimental to the efficacy of the composition containing them.

6-[3-(1-Adamantyl)-4-methoxyphenyl]-2-naphthoic acid (referred to hereinbelow as adapalene) is a naphthoic acid derivative with retinoid and anti-inflammatory properties. This molecule has been the subject of development for the topical treatment of common acne and retinoid-sensitive dermatoses.

Adapalene is sold under the brand name Differin® at a weight concentration of 0.1%, in the form of a solution referred to as an alcoholic lotion, an aqueous gel and a cream. These compositions are intended for treating acne. Patent application FR 2 837 101 describes, for its part, compositions of adapalene at a weight concentration of 0.3%, for treating acne.

Patent application WO 03/055472 moreover describes stable pharmaceutical compositions comprising adapalene and benzoyl peroxide (BPO).

An article by Korkut and Piskin, J. Dermatology, 2005, 32: 169-173, reports the results of a study comparing a “combined” treatment, consisting of an application of adapalene in the evening and an application of BPO in the morning, relative to an application of each of the active principles alone. The authors do not observe any superiority of the “combined” treatment over an 11-week treatment period.

There was nothing to encourage combining these two active agents within the same composition in order to obtain a stable composition of foam type, given that it was usually known that the presence of benzoyl peroxide chemically and physically destabilized this type of composition.

One aim of the present invention is thus to provide novel foam compositions that are particularly suited to topical administration, comprising within the same composition (i.e. in the same vehicle) adapalene and benzoyl peroxide in dispersed form.

The formulation in foam form containing a retinoid and benzoyl peroxide is advantageous for topical treatments, such as that of acne, since it allows an application of the combination of adapalene and benzoyl peroxide on the skin, which is not only pleasant for the patient but also easy, unique and effective. Moreover, this type of formulation has very good cosmeticity for patients.

The majority of the formulation bases for obtaining a foam existing at the present time are in the form of emulsions.

Specifically, patent WO 2009/069006 describes compositions of foam type obtained from emulsions containing only benzoyl peroxide. These compositions especially have a viscosity of less than 8000 cps.

Patent WO 2007/007198 describes compositions of foam type obtained from emulsions containing a retinoid. These compositions have a substantial proportion of organic vehicle representing from 2% to 50% of the total weight of the composition. This high content of organic vehicle is unsuitable for the treatment of acne since it gives a greasy feel.

Specifically, the compositions in foam form existing in the prior art thus have the following drawbacks:
the viscosity of the formulations is unsuitable for easy application. This gives the formulation poor efficacy;
the foams are largely obtained from emulsions. Now, emulsions are compositions which comprise a fatty phase and are thus rich in oils (close to 15% of oils) which are incompatible with the treatment of acne, which, on the contrary, requires refreshing aqueous compositions rather than greasy compositions. These compositions leave a greasy feel on the skin after application.

The existing compositions in foam form therefore do not have all the properties required for the treatment of acne as described previously.

It thus proves to be essential to develop a dermatological composition of foam type obtained from an intermediate composition of gel type and/or a suspension for topical application which affords very good stability, a cosmetically acceptable non-greasy feel (absence of fatty phase), good maintenance of the active agents in dispersed form within the formulation, and a viscosity that enables easy application to the skin, targeted on lesions.

The compositions of foam type obtained from intermediate compositions of gel type according to the invention do not contain a fatty phase and have a viscosity of greater than 8000 cps after preparation at room temperature (25° C.) measured under the conditions defined in example 1 of the present patent application (“Example 1: Characterization of the intermediate formulations of gel and suspension type”).

The compositions of foam type obtained from intermediate compositions of suspension type according to the invention do not contain a fatty phase and have a viscosity of between 8000 cps and 32 000 cps after preparation at room temperature (25° C.) measured under the conditions defined in example 1 of the present patent application (“Example 1: Characterization of the intermediate formulations of gel and suspension type”).

Hereinbelow in the present patent application, the terms “intermediate composition”, “gel”, “composition of gel type”, “intermediate formulation”, “formulation of gel type”, “suspensions”, “composition of suspension type” and “formulation of suspension type” will be used indiscriminantly to denote the intermediate composition leading to the production of the foam composition according to the invention. The terms “composition” and “composition of foam type” and “foam” represent the final composition in foam form.

In the intermediate compositions according to the present invention, the active agents are present in a dispersed form.

The term “gel” means a semi-solid preparation containing a gelling agent which gives rigidity to a solution or to a colloidal dispersion (Lucinda Buhse et al., “Topical drug classification”, International Journal of Pharmaceutics, 2005 (295), 101-112).

The term “suspension” means a liquid preparation containing solid particles dispersed in a liquid vehicle which is compatible for cutaneous application (CDER Data Standards Manual, version 008, Apr. 14, 1992). A liquid flows with little or no external forces and displays newtonian or pseudoplastic behavior (Lucinda Buhse et al., “Topical drug classification”, International Journal of Pharmaceutics, 2005 (295), 101-112).

The Applicant has in particular prepared a foam from an intermediate composition of gel type comprising:
adapalene and benzoyl peroxide,
water,
at least one gelling agent and/or pH-independent gelling agent,
at least one surfactant,
at least one wetting agent,
optionally, a chelating agent,
optionally, at least one humectant and/or emollient,
optionally, one or more additives,
   said adapalene and said benzoyl peroxide being in dispersed form in said composition.

The Applicant has also prepared a foam from an intermediate composition of suspension type comprising:
adapalene and benzoyl peroxide,
water,
at least one gelling agent and/or pH-independent gelling agent,
at least one suspension agent and/or viscosity
enhancer,
at least one surfactant,
at least one wetting agent,
optionally, a chelating agent,
optionally, at least one humectant and/or emollient,
optionally, one or more additives,
   said adapalene and said benzoyl peroxide being in dispersed form in said composition.

The combination of adapalene and benzoyl peroxide is preferably a fixed combination, i.e. a combination whose active principles are combined in fixed doses within the same vehicle (single formulation) delivering them together at the point of application.

In the gels and suspensions according to the invention, adapalene is used at concentrations of between 0.001% and 10% by weight relative to the total weight of the intermediate composition, preferably at concentrations of between 0.01% and 5%, more preferentially between 0.05% and 0.5% and most preferentially from 0.1% to 0.3% by weight relative to the total weight of the intermediate composition.

Benzoyl peroxide may be used either in free form or in an encapsulated form, for example in a form adsorbed onto or absorbed into any porous support. It may be, for example, benzoyl peroxide encapsulated in a polymeric system consisting of porous microspheres, for instance microsponges sold under the name Microsponges P009A Benzoyl peroxide by the company Cardinal Health.

In the gels and suspensions according to the invention, benzoyl peroxide is used at concentrations of between 1% and 10% by weight relative to the total weight of the intermediate composition, preferably at concentrations of between 2% and 7% and more preferentially between 2.5% and 5% by weight relative to the total weight of the intermediate composition.

Advantageously, the particle size of the adapalene is such that at least 90% numerically of the particles have a diameter less than 10 μm and at least 99% numerically of the particles have a diameter less than 50 μm.

Advantageously, the particle size of the benzoyl peroxide is such that at least 100% numerically of the particles have a diameter less than 100 μm and preferably at least 90% numerically of the particles have a diameter less than 20 μm and at least 99% numerically of the particles have a diameter less than 50 μm. The particle sizes are preferably measured by optical microscopy.

Preferably, the adapalene and the benzoyl peroxide are in dispersed form.

According to the invention, the term “active agent in dispersed form” means an active principle in the form of solid particles, suspended in a given vehicle. Such particles preferably have a size greater than 10 μm.

The suspending power for dispersed active agents such as adapalene and benzoyl peroxide of our compositions of gel and suspension type is optimized by means of the addition of at least one gelling agent and in the presence or absence of at least one suspension agent and/or viscosity enhancer.

The aqueous phase of the gel or of the suspension may be present in a content of between 40% and 90% by weight and preferably between 65% and 85% by weight relative to the total weight of the intermediate composition.

The gelling agent(s) and/or pH-independent gelling agent(s) present in the gel or suspension have the role of increasing the viscosity of the aqueous phase. This makes it possible especially to improve the stabilization of this phase and its binding nature, which leads both to good homogeneity of the distribution of the active agents in the intermediate composition and to the production of foams having the desired texture and stability. As nonlimiting examples, the gelling agent(s) and/or pH-independent gelling agent(s) may be chosen especially from:
“electrolyte-insensitive” carbomers sold, as nonlimiting examples, under the name Carbopol Ultrez-20®, Carbopol 1382® or Carbopol ETD 2020® by the company Noveon, and acrylates/C10-30 alkyl crosspolymer sold under the name Pemulen TR-1® or Pemulen TR-2® by the company Noveon;
polysaccharides, nonlimiting examples including the xanthan gum sold under the name Xantural 180® by the company Kelco or Satiaxane UCX911® by the company Cargill, the guar gum sold under the name N-Hance® by the company IMCD, the locust bean gum sold under the name Viscogum® by the company Cargill, gum tragacanth and extracts of quince seeds; alginates such as the sodium alginate sold under the name Satialgine® by the company Cargill;
modified starches such as the modified potato starch sold under the name Structure Solanace® or mixtures thereof;
celluloses and derivatives thereof, for instance the hydroxyethylcellulose sold under the name Natrosol 250HHX® by the company IMCD, the methylcellulose sold under the name Benecel® by the company IMCD, the carboxymethylcellulose sold under the name Blanose 7H4F® by the company IMCD, the hydroxypropylmethylcellulose sold under the name Methocel E4M® by the company Dow Chemicals, and the hydroxypropylcellulose sold under the name. Klucel HF® by the company IMCD;
polyvinyl alcohol, for instance the polyvinyl alcohol 40-88® sold by the company Merck;
the Polyquaternium family, for instance the Polyquaternium-10® sold under the name Celquat SC-240C® by the company National Starch;
acrylic polymers coupled to hydrophobic chains, such as the PEG-150/decyl/SMDI copolymer sold under the name Aculyn 44® (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%));
polyacrylamides such as the acrylamide/sodium acryloyldimethyltaurate copolymer/isohexadecane/polysorbate 80 mixture sold under the name Sepineo P600® (or Simulgel 600PHA®) by the company SEPPIC, the polyacrylamide/C13-14 isoparaffin/laureth-7 mixture, for instance the product sold under the name Sepigel 305® by the company SEPPIC, the hydroxyethyl acrylate/sodium acryloyldimethyltaurate copolymer mixture sold under the name Sepinov EMT 10® by the company SEPPIC; and
mixtures of these compounds.

The gelling agent and/or pH-independent gelling agent as described above may be used at preferential concentrations ranging from 0.1% to 10% by weight and more preferentially ranging from 0.2% to 5% by weight relative to the total weight of the intermediate composition.

Preferred gelling agents that may be mentioned include polysaccharides such as xanthan gum (Xantural 180®), polyacrylamides such as the acrylamide/sodium acryloyldimethyltaurate copolymer/isohexadecane/polysorbate 80 mixture (Sepineo P600® (or Simulgel 600PHA®)) and the hydroxyethyl acrylate/sodium acryloyldimethyltaurate copolymer mixture (Sepinov EMT 10®).

The suspension agent(s) optionally present in the intermediate composition have the role of maintaining in suspension the active agents present in the compositions without, however, increasing the viscosity of said compositions. Examples that may be mentioned include:
the cellulose family, including, inter alia, microcrystalline cellulose and sodium carboxymethylcellulose sold under the name Avicel CL-611® by the company FMC Biopolymer;
the silica family, including, inter alia, Aerosil 200® Pharma and Aerosil R972® sold by the company Evonik;
the polysaccharide family, including, inter alia, Sclerotium rolfsii sold under the name Amigel® by the company Alban Muller, the xanthan gum-locust bean gum combination sold under the name XPV-SG 600® by the company Cargill;
the carrageenan family, in particular divided into four main families: κ, λ, β, ω, such as the Viscarin® products (nonlimiting examples include Viscarin GP-379NF® and Viscarin GP-209NF®) and the Gelcarin® products (a nonlimiting example is Gelcarin GP-379NF®) sold by the company IMCD;
the clay family, including, inter alia, magnesium aluminum silicate such as Veegum K® sold by the company Lavollee Chimie or bentones such as Veegum HS® sold by the company Lavollee Chimie;
the salt family, including, inter alia, the sodium chloride sold by the company Merck;
and mixtures thereof.

The suspension agent and/or viscosity enhancer as described above may be used at preferential concentrations ranging from 0.1% to 10% by weight and more preferentially ranging from 0.2% to 5% by weight relative to the total weight of the intermediate composition.

Preferred suspension agents and/or viscosity enhancers that may be mentioned include microcrystalline cellulose and sodium carboxymethylcellulose sold under the name Avicel CL-611®, carrageenans, for instance Viscarin GP-209NF®, clays, for instance Veegum HS®, and polysaccharides, for instance Amigel®.

The gels and suspensions of the present invention contain surfactants, which are amphiphilic molecules, which will make it possible to form the foam and to stabilize it (A. Arzhavitina, “Foams for pharmaceutical and cosmetics application”, International Journal of Pharmaceutics, 394 (2010), 1-17).

Specifically, surfactants are amphiphilic compounds which bear a hydrophobic part that has affinity for oil and a hydrophilic part that has affinity for water, thus creating a link between the two phases. The polarity of the surfactant is defined by the HLB (hydrophilic-lipophilic balance).

A high HLB indicates that the hydrophilic fraction is predominant, and, conversely, a low HLB indicates that the lipophilic part is predominant. By way of example, HLB values greater than about 10 correspond to hydrophilic surfactants.

Surfactants may be classified, according to their structure, under the generic terms “ionic” (anionic, cationic or amphoteric) or “nonionic”. Nonionic surfactants are surfactants that do not dissociate into ions in water and are thus insensitive to pH variations.

The surfactants present in the intermediate composition provide a surface modification to interfaces of liquid/gas type, which ensures the formation of the foam (Dominique Langevin, “Aqueous foams: a field of investigation at the frontier between chemistry and physics”, ChemPhysChem., 2008 (9), 510-522) and stabilizes the film surrounding each foam bubble (Tim Kealy, Alby Abram, Richard Buchta, “The rheological properties of a pharmaceutical foam: implications for use”, International Journal of Pharmaceutics, 2008 (355), 67-80).

Nonlimiting examples of anionic surfactants that may be mentioned include sodium lauryl sulfate (the sodium lauryl sulfate sold under the name Texapon K12 P PH® by the company Cognis), and ammonium or triethanolamine lauryl sulfate, sodium lauryl ether sulfate (the sodium laureth sulfate sold under the name Texapon N70® by the company Cognis), and magnesium, ammonium or TEA (triethylamine) lauryl ether sulfate, the sodium lauroyl sarcosinate sold under the name Protelan LS9011® by the company Zschimmer & Schwartz, sodium olefin sulfonates, sodium sulfoacetates, sulfosuccinates or taurates, and sodium cocoyl glutamate & disodium cocoyl glutamate sold under the name Amisoft CS-22® by the company Ajinomoto.

Nonlimiting examples of cationic surfactants that may be mentioned include quaternary ammoniums, alkylpyridinium chlorides, alkylammonium saccharinates and amine oxides.

Nonlimiting examples of amphoteric surfactants that may be mentioned include betaines and derivatives thereof, for instance cocamidopropylbetaine sold under the name Amonyl 380 BA® by the company SEPPIC, cocoylbetaine sold under the name Amonyl 265BA® by the company SEPPIC or Dehyton AB 30® by the company Cognis, and disodium cocoamphoacetate sold under the name Rewoteric AM2 C NM® by the company Evonik.

Nonlimiting examples of nonionic surfactants that may be mentioned include sorbitan esters such as POE(20) sorbitan monooleate, sold under the name Tween 80®, POE(20) sorbitan monostearate, sold under the name Tween 60®, sorbitan monostearate sold under the name Span 60® by the company Uniqema, glycerol esters such as the glyceryl monostearate sold under the name Cutina GMSVPH® by the company Cognis, polyethylene glycol esters such as PEG-6 isostearate sold under the name Olepal isostearique® by the company Gattefosse, fatty alkyl ethers such as POE(21) stearyl ether sold under the name Brij 721® by the company Uniqema or Ceteareth-20 sold under the name Eumulgin B2PH® by the company Cognis, polyoxyalkylene glycol esters such as glyceryl stearate and PEG-100 stearate sold under the name Arlacel 165 Flakes® by the company Uniqema, PEG 6 stearate and PEG 32 stearate sold under the name Tefose 1500® by the company Gattefosse, sucroesters with sucrose laurate sold under the name Surfhope D-1216® or Surfhope C1215L® by the company Gattefosse, or the mixture of aqua (and) sucrose laurate (and) alcohol sold under the name Sisterna L70C® by the company Gattefosse, PEG-40 hydrogenated castor oil sold under the name Eumulgin HRE40PH® by the company Cognis, decyl glucoside sold under the name Oramix NS10® by the company SEPPIC, and caprylyl capryl glucoside sold under the name Oramix CG110® by the company SEPPIC.

Irrespective of its nature, the surfactant as described above is preferably included in a content of between 0.2% and 15% by weight and preferably between 0.5% and 10% by weight relative to the total weight of the intermediate composition.

To obtain a foam with optimum properties, the surfactants that are particularly preferred are chosen from nonionic surfactants (Tween 80®, Surfhope C1215L®, Sisterna L70C®, Oramix NS10®, Eumulgin HRE40PH®).

The composition of gel or suspension type according to the invention also comprises at least one wetting agent. The role of wetting agents is to reduce the surface tension and to allow greater spreading of the liquid. A wetting agent that may preferentially have an HLB from 10 to 14 is used, without this list being limiting. Among the wetting agents that may be used according to the invention, mention may be made of compounds from the Poloxamer family, including Poloxamer 124 sold under the name Synperonic PE/L44® by the company Uniqema or Lutrol L44® sold by the company BASF, Poloxmer 182 sold under the name Synperonic PE/L62® by the company Uniqema or Lutrol L62® by the company BASF, and compounds of the glycol family, including propylene glycol, dipropylene glycol, propylene glycol dipelargonate, lauroglycol and ethoxydiglycol.

Preferably, wetting agents are in liquid form so as to be readily incorporated into the composition of gel or suspension type without it being necessary to heat it.

The wetting agent as described above may be used at preferential concentrations ranging from 0.05% to 10% by weight and more preferentially ranging from 0.1% to 8% by weight relative to the total weight of the intermediate composition.

The wetting agents that are particularly preferred are propylene glycol and Lutrol L44® sold by the company BASF.

Among the chelating agents that are optionally present in the intermediate composition according to the invention, nonlimiting examples that may be mentioned include ethylenediaminetetraacetic acid (EDTA), diethylenetriaminepentaacetic acid (DTPA), ethylenediaminebis(β-hydroxyphenylacetic acid) (EDDHA), hydroxy-2-ethylenediaminetriacetic acid (HEDTA), ethyldiaminebis(O-hydroxy-p-methylphenyl)acetic acid (EDDHMA) and ethylenediaminebis(5-carboxy-2-hydroxy-phenyl)acetic acid (EDDHMA).

The chelating agent as described above may be used at preferential concentrations ranging from 0% to 1.5% by weight and more preferentially ranging from 0% to 1% by weight relative to the total weight of the intermediate composition. When the chelating agent is present in the composition, its concentration is preferably between 0.01% and 1%.

A preferred chelating agent that may be mentioned is ethylenediaminetetraacetic acid (EDTA) sold in particular under the name Titriplex III®.

The intermediate compositions of gel or suspension type according to the invention may also contain humectants and/or emollients, the role of which is to moisturize the skin and to facilitate the application of the formulation. Humectants and/or emollients that are preferentially used, without this list being limiting, include compounds such as glycerol and sorbitol, sugars (for example glucose or lactose), PEGs (for example Lutrol E400), urea, amino acids (for example serine, citrulline, arginine, asparagine or alanine). These agents are taken alone or in combination in the composition.

The humectant and/or emollient as described above may be used at preferential concentrations ranging from 0 to 20% by weight and more preferentially ranging from 0 to 15% by weight relative to the total weight of the intermediate composition. When the humectant and/or emollient is present in the composition, its concentration is preferably between 0.01% and 15%.

A preferred humectant and/or emollient that may be mentioned is glycerol.

The intermediate compositions according to the invention may also optionally comprise any additive normally used in the cosmetic or pharmaceutical field, such as neutralizers of common mineral or organic acid or base type, sunscreens, antioxidants, fillers, electrolytes, preserving agents, dyes, fragrances, essential oils, cosmetic active agents, moisturizers, vitamins, essential fatty acids, sphingolipids, self-tanning compounds, calmatives and skin-protecting agents such as allantoin, pro-penetrating agents, or a benzoyl peroxide stabilizer, or a mixture thereof. Needless to say, a person skilled in the art will take care to select this or these optional additional compound(s), and/or the amount thereof, such that the advantageous properties of the composition according to the invention are not, or are not substantially, adversely affected.

These additives as described above may be used at preferential concentrations ranging from 0% to 20% by weight and more preferentially ranging from 0 to 15% by weight relative to the total weight of the intermediate composition. When the additive is present in the composition, its concentration is preferably between 0.01% and 15%.

A person skilled in the art will take care to select the excipients constituting the intermediate compositions according to the invention according to the desired galenical form and such that the advantageous properties of the intermediate composition according to the invention are respected.

The invention thus relates to a pharmaceutical composition based on adapalene or a salt thereof and benzoyl peroxide, characterized in that it is in the form of a foam obtained from an intermediate composition of gel or suspension type.

More particularly, the invention relates to a pharmaceutical composition characterized in that it is in the form of a foam obtained from an intermediate composition of gel or suspension type, said composition comprising:
between 60% and 98% by weight and preferentially between 80% and 96% by weight, relative to the total weight of the composition, of a gel or suspension,
between 2% and 40% by weight and preferentially between 4% and 20% by weight, relative to the total weight of the composition, of at least one propellant gas.

In one particular embodiment according to the invention, the intermediate composition in gel form comprises (percentage expressed on a weight basis relative to the total weight of the composition of gel type):
(a) 0.001% to 10% and preferentially from 0.01% to 5% of adapalene and 1% to 10% and preferentially from 2% to 7% of benzoyl peroxide in combination;
(b) 40% to 90% of water and preferentially from 65% to 85% of water;
(c) 0.1% to 10% and preferentially from 0.2% to 5% of at least one gelling agent and/or pH-independent gelling agent for the aqueous phase;
(d) 0.2% to 15% and preferentially from 0.5% to 10% of at least one surfactant;
(e) 0.05% to 10% and preferentially from 0.1% to 8% of at least one wetting agent;
(f) 0% to 1.5% and preferentially from 0% to 1% of a chelating agent;
(g) 0% to 20% and preferentially from 0% to 15% of at least one humectant and/or emollient;
(h) 0% to 20% and preferentially from 0% to 15% of one or more additives; said adapalene and said benzoyl peroxide being in dispersed form in said gel.

In another particular embodiment according to the invention, the intermediate composition in suspension form comprises (percentage expressed on a weight basis relative to the total weight of the composition of suspension type):
(a) 0.001% to 10% and preferentially from 0.01% to 5% of adapalene and 1% to 10% and preferentially from 2% to 7% of benzoyl peroxide in combination;
(b) 40% to 90% of water and preferentially from 65% to 85% of water;
(c) 0.1% to 10% and preferentially from 0.2% to 5% of at least one gelling agent and/or pH-independent gelling agent for the aqueous phase;
(d) 0.1% to 10% and preferentially from 0.2% to 5% of at least one suspension agent and/or viscosity enhancer;
(e) 0.2% to 15% and preferentially from 0.5% to 10% of at least one surfactant;
(f) 0.05% to 10% and preferentially from 0.1% to 8% of at least one wetting agent;
(g) 0% to 1.5% and preferentially from 0% to 1% of a chelating agent;
(h) 0% to 20% and preferentially from 0% to 15% of at least one humectant and/or emollient;
(i) 0% to 20% and preferentially from 0% to 15% one or more additives;
said adapalene and said benzoyl peroxide being in dispersed form in said suspension.

According to a preferred mode, the invention relates to a pharmaceutical composition based on adapalene and benzoyl peroxide in combination, characterized in that it is in the form of a foam obtained from an intermediate composition of gel or suspension type which comprises:
between 80% and 96% by weight, relative to the total weight of the composition, of a gel or suspension,
between 4% and 20% by weight, relative to the total weight of the composition, of at least one propellant gas,
said gel comprising (percentage expressed on a weight basis relative to the total weight of the composition of gel type):
(a) 0.01% to 5% of adapalene and 2% to 7% of benzoyl peroxide in combination;
(b) 65% to 85% of water;
(c) 0.2% to 5% of at least one gelling agent and/or pH-independent gelling agent for the aqueous phase;
(d) 0.5% to 10% of at least one surfactant;
(e) 0.1% to 8% of at least one wetting agent;
(f) 0% to 1% of a chelating agent;
(g) 0% to 15% of at least one humectant and/or emollient;
(h) 0% to 15% of one or more additives;
said adapalene and said benzoyl peroxide being in dispersed form in said gel.
Said suspension comprising (percentage expressed on a weight basis relative to the total weight of the composition of suspension type):
(a) 0.01% to 5% of adapalene and 2% to 7% of benzoyl peroxide in combination;
(b) 65% to 85% of water;
(c) 0.2% to 5% of at least one gelling agent and/or pH-independent gelling agent for the aqueous phase;
(d) 0.2% to 5% of at least one suspension agent and/or viscosity enhancer;
(e) 0.5% to 10% of at least one surfactant;
(f) 0.1% to 8% of at least one wetting agent;
(g) 0% to 1% of a chelating agent;
(h) 0% to 15% of at least one humectant and/or emollient;
(i) 0% to 15% of one or more additives;
(j) 4% to 20% of at least one propellant gas;
said adapalene and said benzoyl peroxide being in dispersed form in said suspension.

The invention also relates to the use of the novel composition of foam type as described previously in cosmetics and dermatology.

By virtue of the keratolytic, bactericidal and anti-inflammatory activity of benzoyl peroxide and of the pronounced activity of adapalene in the fields of cell differentiation and proliferation, the compositions or the gels and suspensions of the invention are particularly suitable for use in the following therapeutic fields:
1) for treating dermatological complaints associated with a keratinization disorder relating to cell differentiation and proliferation, especially for treating common acne, comedones, polymorphs, acne rosacea, nodulocystic acne, acne conglobata, senile acne, and secondary acnes such as solar acne, medication-related acne or occupational acne, and hidradenitis suppurativa;
2) for treating other types of keratinization disorders, especially ichthyosis, ichthyosiform conditions, Darier's disease, palmoplantar keratoderma, leukoplakia and leukoplakiform conditions, and cutaneous or mucous (oral) lichen;
3) for treating other dermatological complaints associated with a keratinization disorder with an inflammatory and/or immunoallergic component, and especially all forms of psoriasis whether cutaneous, mucous or ungual, and even psoriatic rheumatism, or cutaneous atopy, such as eczema, or respiratory atopy or alternatively gingival hypertrophy; the compounds may also be used in certain inflammatory complaints not presenting any keratinization disorder, such as folliculitis;
4) for treating all dermal or epidermal proliferations, whether benign or malignant, and whether of viral origin or otherwise, such as common warts, flat warts, molluscum contagiosum and verruciform epidermodysplasia, oral or florid papillomatoses, and proliferations that may be induced by ultraviolet radiation, especially in the case of actinic keratoses;
5) for combating sebaceous function disorders, such as the hyperseborrhea of acne or simple seborrhea;
6) in the treatment of dermatological or general complaints with an immunological component;
7) in the treatment of dermatological complaints associated with an inflammation or infection of the tissues surrounding the hair follicles, especially caused by microbial colonization or infection, especially impetigo, seborrheic dermatitis, folliculitis or sycosis barbae, or involving any other bacterial or fungal agent.

The compositions or gels and suspensions according to the invention are particularly suitable for preventively or curatively treating common acne.

The compositions according to the invention also find an application in cosmetics, in particular for the treatment of acne-prone skin and for combating the greasy appearance of the skin or the hair.

Preferentially, said compositions according to the invention are administered topically.

A subject of the invention is also a process for preparing a composition of gel or suspension type as described previously.

The main process for preparing the intermediate composition of gel or suspension type according to the invention comprises, by way of example, the following steps:

### Step a: Preparation of the Active Phase 1:

Mixing of purified water and of the active principle 1 (adapalene) with at least one wetting agent until said adapalene is fully dispersed, so as to obtain the active phase 1;

### Step b: Preparation of the Active Phase 2:

Mixing of purified water and of the active principle 2 (benzoyl peroxide) with at least one wetting agent until said benzoyl peroxide is fully dispersed, so as to obtain the active phase 2;

### Step c: Preparation of the Aqueous Phase

Purified water and the gelling agent(s) and/or the pH-independent gelling agent(s) or the hydrophilic surfactants and optionally the suspension agent(s) and/or viscosity enhancer(s), the chelating agent, the preserving agent(s), the stabilizer(s) and the humectant(s) and/or emollient(s) are placed in a beaker with stirring, if necessary with heating.

### Step d: Mixing of Two Active Phases

The two active phases obtained previously are mixed together, and stirring is continued until fully homogenized.

### Step e: Preparation of the Gel or Suspension

The single active phase obtained in step d) is then introduced with stirring into the aqueous phase.

### Step f: Addition of Polyacrylamide (Optional)

Polyacrylamide is introduced into the gel or suspension with stirring. The stirring is maintained until fully homogenized.

### Step g: Neutralization Step (Optional)

The gelling-agent neutralizer is introduced if necessary into the gel or suspension.

Step h: Water adjustment (optional)

If necessary, a water adjustment is performed.

The additives will be added, if present in the gel or the suspension, to the aqueous phase.

More specifically, an alternative process may be envisaged: The alternative process for preparing the intermediate composition according to the invention comprises, by way of example, the following steps:

The active principles are mixed together in the first step of the process described above; thus, steps a) and b) are replaced with step a′):

a′) preparation of the single active phase comprising the two active agents.

The process is then continued as described from step c) with omission of step d).

According to yet another specific aspect, a subject of the invention is a process for preparing a composition in foam form based on the combination of adapalene and benzoyl peroxide, by mixing a gel or suspension with at least one propellant gas.

Foams are defined as a dispersion of a gas in liquid or a solid (A. Arzhavitina, “Foams for pharmaceutical and cosmetics application”, International Journal of Pharmaceutics, 394 (2010), 1-17).

The European pharmacopea 6th edition 2010 describes a “medicated foam” as being a preparation formed by the dispersion of a large volume of gas in a liquid preparation generally containing one or more active principles, at least one surfactant for ensuring their formation, and various other excipients.

The American pharmacopea USP Chapter 1151 lists foams in the section “Aerosol foam”. This is a composition containing one or more active principles, one or more surfactants, aqueous or non-aqueous liquids, and propellants.

The compositions in foam form of the present invention are obtained by introducing the intermediate composition of gel and/or suspension type into an aerosol container containing at least one propellant gas under pressure. The aerosol is formed from three components “Pharmaceutical Dosage forms, USP 1151”:
the leaktight can;
the valve for stoppering it and for placing the container in communication with the atmosphere to dispense the product;
the diffuser or pushbutton comprises the valve aperture and makes it possible to vary the flow rate.

By liberating the formulation of gel or suspension type from the container by means of the pushbutton, a foam is obtained.

The aerosol container used in the context of this embodiment is preferably a container of shaving foam can type, namely a closed container under pressure, comprising an outlet nozzle connected to the gel or suspension and containing at least one propellant gas, a valve and a pushbutton suitable for dispensing the foam.

The aerosol thus differs from certain pump sprayers that act only by the action of a mechanical spring (absence of propellant gas). It should be noted that an aerosol always contains a propellant that flushes out and disperses the product (Martini M. C., Esthétique-cosmétique, volume 2, “Cosmétologie”, Editions Masson, Paris, 2002).

The “propellant gases” that may be used in the present invention are of two types: compressed gases, liquefied gases.

Compressed gases are gaseous at room temperature. Examples that may be mentioned include nitrogen, carbon dioxide and nitrous oxide, and mixtures thereof.

Liquefied gases are liquid at room temperature. Examples that may be mentioned include butane, propane and isobutane, and mixtures thereof.

The propellant gases used according to the present invention are used in proportions ranging from 2% to 40% and preferentially ranging from 4% to 20% by weight of the composition.

According to a particular aspect, the aerosol containers for dispensing a foam, comprising a gel or suspension and at least one propellant under pressure, constitute another specific subject of the present invention.

The invention and the advantages arising therefrom will emerge more clearly from the following implementation examples. These examples are, however, in no way limiting.

Examples of preparation of intermediate formulations of gel and suspension type, and examples of compositions of foam type according to the invention, are thus described below. Similarly, the tests for characterizing the intermediate compositions and the foams are also defined.

### EXAMPLE 1

### Characterization of the Intermediate Compositions of Gel and Suspension Type

The physical stability of the intermediate formulations of gel or suspension type is controlled by a macroscopic and microscopic observation, conserved at room temperature (RT) and 40° C. after T+1 month or T+2 months or T+3 months.

At room temperature and 40° C., the macroscopic observation makes it possible to ensure the physical integrity of the products.

At room temperature, the microscopic observation makes it possible to evaluate the quality of dispersion of the two active agents. Adapalene is observed in fluorescent light, whereas benzoyl peroxide is observed in polarized light.

Characterization of the gel and of the suspension is completed by a viscosity measurement and by establishing a rheological profile.

The apparent viscosity of the gel and of the suspension is established using Brookfield RVDVII+ and LVDVII+ viscometers at room temperature (25° C.)

The viscosity ranges that are measurable with these two types of Brookfield viscometer are as follows:
RVDVII+ viscometer: 100 cP-40 McP
LVDVII+ viscometer: 15 cP-6 McP

This apparent viscosity measurement yields information regarding the viscosity of the gel or of the suspension at rest (in the packaging),

The establishment of the rheological profile of the gel or of the suspension makes it possible to describe the rheological properties of the formulation, especially its flow threshold.

For the flow threshold measurement, a Haake VT550 rheometer with an SVDIN measuring spindle is used.

The rheograms are produced at 25° C. and at an imposed speed of 0 to 100 s⁻¹. The viscosity values are noted at the shear values and at constant shear rates of 4 s⁻¹, 20 s⁻¹ and 100 s⁻¹ (γ), and by measuring the shear stress. The term “flow threshold” (τ₀ expressed in Pascals) means the force (minimum shear stress) required to overcome the cohesion forces of Van der Waals type and to bring about flow. The flow threshold is likened to the value found at the shear rate of 4 s⁻¹.

The chemical stability is determined by an HPLC assay of the adapalene and by an iodometric assay for benzoyl peroxide.

The results are expressed as a percentage relative to the Label Claim (LC) (theoretical content of adapalene and of benzoyl peroxide).

These physical and chemical tests will make it possible to ensure the good stability over time of the various gels and suspensions and thus of the foams obtained according to the invention.

### EXAMPLE 2

### Characterization of the Foams

The physical stability of the foams obtained is also characterized by means of the tests presented below:
determination of the organoleptic characteristics (aspect, color, odor),
characterization of the texture (thick, fluid, greasy, non-greasy),
characterization of the spreadability (classified from 1 (spreads easily) to 5 (very difficult to spread)),
The quality of the foam leaving the container is evaluated according to a classification on a scale from 1 to 5, with “1” representing a foam with fine bubbles and “5” representing a foam with large bubbles,
The foam density measurement is performed according to the protocol described in the European Pharmacopea 6th edition 2010:
   Protocol: Maintain the container at a temperature of 25° C. for at least 24 hours. While avoiding heating the container, fit a rigid tube 70 mm to 100 mm long and with an inside diameter of about 1 mm onto the pushbutton, shake the container to homogenize the liquid phase, and expel 5 ml to 10 ml of foam. Tare a flat-bottomed crystallizing dish with a volume of about 60 ml and a height of about 35 mm. Place the extremity of the rigid tube in the angle at the bottom of the crystallizing dish, and, to fill it uniformly, press the pushbutton while effecting a circular motion. After total expansion of the foam, level it off by scraping off the excess using a blade. Weigh. Determine the mass of the same volume of water R by filling the same crystallizing dish with water R. The foam density is equal to the ratio: m/e
   m=mass of the foam sample, in grams
   e=mass of the same volume of water R, in grams
   Take three measurements. None of the individual values differs from the mean value by more than 20%.
The foam expansion time is determined according to the protocol described in the European Pharmacopea 6th edition 2010:
   Protocol: The apparatus consists of a 50 ml burette, with an inside diameter of 15 mm, with graduations of 0.1 ml, fitted with a 4 mm one-way tap. The graduation corresponding to 30 ml is at 210 mm at least from the axis of the tap. The bottom of the burette is connected, via a plastic tube with a maximum length of 50 mm and an inside diameter of 4 mm, to the foam-generating container fitted with a pushbutton adapted to this connection. Maintain the container at a temperature of 25° C. for at least 24 hours. While avoiding heating it, shake the container to homogenize the liquid phase, and expel 5 ml to 10 ml of foam. Connect the pushbutton to the outlet of the burette. Press the pushbutton and introduce, in one go, an amount of foam in the region of 30 ml. Close the tap, simultaneously start the chronometer and read the volume of foam contained in the burette. Read every ten seconds the volume that increases up to the maximum volume. Take three measurements. None of the times necessary to obtain the maximum volume is greater than 5 minutes.

These tests make it possible to ensure the good stability over time of the various foams obtained.

### EXAMPLE 3

### Formulation of Gel Type Containing 0.1% Adapalene and 2.5% Benzoyl Peroxide

| Constituents | Concentration (%) |
|---|---|
| Adapalene | 0.10 |
| BPO | 2.50 |
| Hydroxyethyl | 2.00 |
| acrylate/sodium | |
| acryloyldimethyltaurate | |
| copolymer | |
| Decyl glucoside | 2.00 |
| Polysorbate 80 | 5.00 |
| Ethylenediaminetetraacetic | 0.05 |
| acid | |
| Sodium docusate | 0.05 |
| Glycerol | 4.00 |
| Propylene glycol | 4.00 |
| Poloxamer 124 | 0.20 |
| Purified water | qs 100% |

Specifications at T0 of the formulation base of gel type:

Characterization at T0

| Macroscopic appearance | Glossy white gel |
|---|---|
| Microscopic appearance | Homogeneous dispersion of |
| | the active agents |
| pH | 4.71 |
| Viscosity (Brookfield | 52000 |
| RVDVII+) in cps | |
| S28/5 RPM | |
| Haake profile | 65/89/150 |

### Physical Stability of the Formulation Base:

| | | T + 1 month | T + 2 months | T + 3 months |
|---|---|---|---|---|
| Macroscopic | RT | In | In | In |
| appearance | | accordance | accordance | accordance |
| | | with T0 | with T0 | with T0 |
| | 40° C. | In | In | In |
| | | accordance | accordance | accordance |
| | | with T0 | with T0 | with T0 |
| Microscopic | RT | In | In | In |
| appearance | | accordance | accordance | accordance |
| | | with T0 | with T0 | with T0 |
| pH | RT | 4.69 | 4.55 | 5.38 |
| Viscosity | RT | 45000 | 45400 | 46300 |
| (Brookfield | | | | |
| RVDVII+) in | | | | |
| cps S28/5 RPM | | | | |
| Haake rheology | RT | 65/89/150 | 68/97/157 | 68/102/158 |
| (4 s⁻¹/20 | | | | |
| s⁻¹/100 s⁻¹) | | | | |

### Chemical Stability

Adapalene:

| Stability | | | | |
|---|---|---|---|---|
| conditions | T0 | T + 1 month | T + 2 months | T + 3 months |
| RT | 99.6% LC | 100.3% LC | 93.8% LC | 99.5% LC |
| 40° C. | NA | 100.2% LC | 97.8% LC | 101.6% LC |

Benzoyl Peroxide:

| Stability | | | | |
|---|---|---|---|---|
| conditions | T0 | T + 1 month | T + 2 months | T + 3 months |
| RT | 100.8% LC | 104% LC | 100.2% LC | 99% LC |
| 40° C. | NA | 96.7% LC | 92% LC | 90.1% LC |

### EXAMPLE 4

### Formulation of Gel Type Containing 0.1% Adapalene and 2.5% Benzoyl Peroxide

| Constituents | Concentration (%) |
|---|---|
| Adapalene | 0.10 |
| BPO | 2.50 |
| Sodium acryloyldimethyltaurate | 3.00 |
| copolymer/isohexadecane/polysorbate 80 | |
| Aqua (and) sucrose laurate (and) | 2.00 |
| alcohol | |
| Glycerol | 4.00 |
| Ethylenediaminetetraacetic acid | 0.05 |
| Propylene glycol | 5.00 |
| Poloxamer 124 | 0.20 |
| Purified water | qs 100% |

Specifications at T0 of the formulation base of gel type:

Characterization at T0

| | |
|---|---|
| Macroscopic appearance | Glossy white gel |
| Microscopic appearance | Homogeneous dispersion of |
| | the active agents |
| pH | 4.38 |
| Viscosity (Brookfield | 79400 |
| RVDVII+) in cps | |
| S29/5 RPM | |
| Haake rheology | 105/134/218 |
| (4 s⁻¹/20 s⁻¹/100 s⁻¹) | |

### Physical Stability of the Formulation Base:

| | | T + 1 month | T + 2 months | T + 3 months |
|---|---|---|---|---|
| Macroscopic | RT | In | In | In |
| appearance | | accordance | accordance | accordance |
| | | with T0 | with T0 | with T0 |
| | 40° C. | In | In | In |
| | | accordance | accordance | accordance |
| | | with T0 | with T0 | with T0 |
| Microscopic | RT | In | In | In |
| appearance | | accordance | accordance | accordance |
| | | with T0 | with T0 | with T0 |
| pH | RT | 4.20 | 4.04 | 3.86 |
| Viscosity | RT | 83400 | 87800 | NR |
| (Brookfield | | | | |
| RVDVII+) in | | | | |
| cps S29/5 RPM | | | | |
| Haake | RT | 113/158/227 | 108/156/249 | 112/165/245 |
| rheology | | | | |
| (4 s⁻¹/20 | | | | |
| s⁻¹/100 s⁻¹) | | | | |

### Chemical Stability

Adapalene:

| Stability | | | | |
|---|---|---|---|---|
| conditions | T0 | T + 1 month | T + 2 months | T + 3 months |
| RT | 112.0% LC | 112.2% LC | 113.0% LC | 112.2% LC |
| 40° C. | NA | 113.7% LC | 114.0% LC | 113.9% LC |

Benzoyl Peroxide:

| Stability | | | | |
|---|---|---|---|---|
| conditions | T0 | T + 1 month | T + 2 months | T + 3 months |
| RT | 101.3% LC | 101% LC | 103.6% LC | 104.0% LC |
| 40° C. | NA | 99.5% LC | 99.0% LC | 96.8% LC |

### EXAMPLE 5

### Formulation of Suspension Type Containing 0.1% Adapalene and 2.5% Benzoyl Peroxide

| Constituents | Concentration (%) |
|---|---|
| Adapalene | 0.10 |
| BPO | 2.50 |
| Microcrystalline cellulose and | 1.50 |
| sodium carboxymethylcellulose | |
| Sodium acryloyldimethyltaurate | 2.00 |
| copolymer/isohexadecane/polysorbate 80 | |
| Sucrose laurate | 3.00 |
| Glycerol | 4.00 |
| Sodium docusate | 0.05 |
| Ethylenediaminetetraacetic acid | 0.05 |
| Propylene glycol | 4.00 |
| Poloxamer 124 | 0.20 |
| Purified water | qs 100% |

Specifications at T0 of the formulation base of gel type:

Characterization at T0

| | |
|---|---|
| Macroscopic appearance | Opaque white suspension, |
| | flows at T0 |
| Microscopic appearance | Homogeneous dispersion of |
| | the active agents |
| pH | 5.45 |
| Haake rheology | 22/33/49 |
| (4 s⁻¹/20 s⁻¹/100 s⁻¹) | |

### Physical Stability of the Formulation Base:

| | | T + 1 month | T + 2 months | T + 3 months |
|---|---|---|---|---|
| Macroscopic | RT | In | In | In |
| appearance | | accordance | accordance | accordance |
| | | with T0 | with T0 | with T0 |
| | 40° C. | In | In | In |
| | | accordance | accordance | accordance |
| | | with T0 | with T0 | with T0 |
| Microscopic | RT | In | In | In |
| appearance | | accordance | accordance | accordance |
| | | with T0 | with T0 | with T0 |
| pH | RT | 5.32 | 5.07 | 4.94 |
| Haake rheology | RT | 25/37/50 | 27/36/52 | 19/28/33 |
| (4 s⁻¹/20 | | | | |
| s⁻¹/100 s⁻¹) | | | | |

### Chemical Stability

Adapalene:

| Stability | | | | |
|---|---|---|---|---|
| conditions | T0 | T + 1 month | T + 2 months | T + 3 months |
| RT | 101.4% LC | 100.2% LC | 100.0% LC | 98.7% LC |
| 40° C. | NA | 98.4% LC | 99.0% LC | 100.8% LC |

Benzoyl Peroxide:

| Stability | | | | |
|---|---|---|---|---|
| conditions | T0 | T + 1 month | T + 2 months | T + 3 months |
| RT | 99.6% LC | 99.7% LC | 101.3% LC | 100.2% LC |
| 40° C. | NA | 96.5% LC | 95.1% LC | 93.0% LC |

### EXAMPLE 6

### Formulation of Suspension Type Containing 0.1% Adapalene and 2.5% Benzoyl Peroxide

| Constituents | Concentration (%) |
|---|---|
| Adapalene | 0.10 |
| BPO | 2.50 |
| Xantural 180 | 0.40 |
| Sodium acryloyldimethyltaurate | 1.00 |
| copolymer/isohexadecane/polysorbate 80 | |
| Bentone | 4.00 |
| Aqua (and) sucrose laurate (and) | 2.00 |
| alcohol | |
| Glycerol | 4.00 |
| Propylene glycol | 4.00 |
| Poloxamer 124 | 0.20 |
| Ethylenediaminetetraacetic acid | 0.05 |
| Purified water | qs 100% |

Specifications at T0 of the formulation base of suspension type:

Characterization at T0

| | |
|---|---|
| Macroscopic appearance | Opaque white suspension, |
| | flows at T0 |
| Microscopic appearance | Homogeneous dispersion of |
| | the active agents |
| pH | 6.98 |
| Viscosity (Brookfield | 31493 |
| LVDVII+) in cps | |
| S64/6 RPM | |
| Haake rheology | 33/51/78 |
| (4 s⁻¹/20 s⁻¹/100 s⁻¹) | |

### Physical Stability of the Formulation Base:

| | | T + 1 | T + 2 | T + 3 |
|---|---|---|---|---|
| | | month | months | months |
| Macroscopic | RT | In | In | In |
| appearance | | accordance | accordance | accordance |
| | | with T0 | with T0 | with T0 |
| | 40° C. | In | In | In |
| | | accordance | accordance | accordance |
| | | with T0 | with T0 | with T0 |
| Microscopic | RT | In | In | In |
| appearance | | accordance | accordance | accordance |
| | | with T0 | with T0 | with T0 |
| pH | RT | 4.92 | 4.71 | 4.53 |
| Viscosity | RT | 31093 | 31893 | NR |
| (Brookfield | | | | |
| LVDVII+) in | | | | |
| cps S64/6 RPM | | | | |
| Haake rheology | RT | 31/45/68 | 32/48/78 | 30/42/64 |
| (4 s⁻¹/20 | | | | |
| s⁻¹/100 s⁻¹) | | | | |

### Chemical Stability

Adapalene:

| Stability | | | | |
|---|---|---|---|---|
| conditions | T0 | T + 1 month | T + 2 months | T + 3 months |
| RT | 107.5% LC | 106.7% LC | 107.0% LC | 105.9% LC |
| 40° C. | NA | 106.9% LC | 104.2% LC | 103.7% LC |

Benzoyl Peroxide:

| Stability | | T + 1 | T + 2 | T + 3 |
|---|---|---|---|---|
| conditions | T0 | month | months | months |
| RT | 103.6% LC | 101.9% LC | 105.0% LC | 103.1% LC |
| 40° C. | NA | 98.4% LC | 96.1% LC | 92.7% LC |

### EXAMPLE 7

### Formulation of Gel Type Containing 0.1% Adapalene and 2.5% Benzoyl Peroxide

| Constituents | Concentration (%) |
|---|---|
| Adapalene | 0.10 |
| BPO | 2.50 |
| Sodium acryloyldimethyltaurate | 2.00 |
| copolymer/isohexadecane/polysorbate 80 | |
| Aqua (and) sucrose laurate (and) | 2.00 |
| alcohol | |
| Glycerol | 4.00 |
| Ethylenediaminetetraacetic acid | 0.05 |
| Propylene glycol | 5.00 |
| Poloxamer 124 | 0.20 |
| Purified water | qs 100% |

Specifications at T0 of the formulation base of gel type:

Characterization at T0

| | |
|---|---|
| Macroscopic appearance | Glossy white gel |
| Microscopic appearance | Homogeneous dispersion of |
| | the active agents |
| pH | 4.58 |
| Viscosity (Brookfield | 28600 |
| LVDVII+) in cps | |
| S64/12 RPM | |
| Haake rheology | 40/63/94 |
| (4 s⁻¹/20 s⁻¹/100 s⁻¹) | |

### Physical Stability of the Formulation Base:

| | | T + 1 | T + 2 | T + 3 |
|---|---|---|---|---|
| | | month | months | months |
| Macroscopic | RT | In | In | In |
| appearance | | accordance | accordance | accordance |
| | | with T0 | with T0 | with T0 |
| | 40° C. | In | In | In |
| | | accordance | accordance | accordance |
| | | with T0 | with T0 | with T0 |
| Microscopic | RT | In | In | In |
| appearance | | accordance | accordance | accordance |
| | | with T0 | with T0 | with T0 |
| pH | RT | 4.20 | 4.10 | 4.00 |
| Viscosity | RT | 24245 | 19596 | 18546 |
| (Brookfield | | | | |
| LVDVII+) in | | | | |
| cps S64/12 RPM | | | | |
| Haake rheology | RT | 40/62/99 | 37/60/106 | 42/66/113 |
| (4 s⁻¹/20 | | | | |
| s⁻¹/100 s⁻¹) | | | | |

### Chemical Stability

Adapalene:

| Stability | | | | |
|---|---|---|---|---|
| conditions | T0 | T + 1 month | T + 2 months | T + 3 months |
| RT | 96.8% LC | 98.4% LC | NA | 97.2% LC |
| 40° C. | NA | 98.5% LC | NA | 96.7% LC |

Benzoyl Peroxide:

| Stability | | | | |
|---|---|---|---|---|
| conditions | T0 | T + 1 month | T + 2 months | T + 3 months |
| RT | 101.4% LC | 100.4% LC | NA | 99.6% LC |
| 40° C. | NA | 98.9% LC | 96.7% LC | 95% LC |

### EXAMPLE 8

### Formulation of Suspension Type Containing 0.1% Adapalene and 2.5% Benzoyl Peroxide

| Constituents | Concentration (%) |
|---|---|
| Adapalene | 0.10 |
| BPO | 2.50 |
| Sodium acryloyldimethyltaurate | 3.00 |
| copolymer/isohexadecane/polysorbate 80 | |
| Carrageenan | 1.00 |
| PEG-40 hydrogenated castor oil | 2.00 |
| Glycerol | 4.00 |
| Ethylenediaminetetraacetic acid | 0.05 |
| Propylene glycol | 4.00 |
| Poloxamer 124 | 0.20 |
| Purified water | qs 100% |

Specifications at T0 of the formulation base of suspension type:

Characterization at T0

| | |
|---|---|
| Macroscopic appearance | Fluid opaque white |
| | suspension, flows at T0 |
| Microscopic appearance | Homogeneous dispersion of |
| | the active agents |
| pH | 4.81 |
| Viscosity (Brookfield | 18580 |
| LVDVII+) in cps | |
| S63/6 RPM | |
| Haake rheology | 38/96/180 |
| (4 s⁻¹/20 s⁻¹/100 s⁻¹) | |

### Physical Stability of the Formulation Base:

| | | T + 1 month | T + 2 months |
|---|---|---|---|
| Macroscopic appearance | RT | In | In |
| | | accordance | accordance |
| | | with T0 | with T0 |
| | 40° C. | In | In |
| | | accordance | accordance |
| | | with T0 | with T0 |
| Microscopic appearance | RT | In | In |
| | | accordance | accordance |
| | | with T0 | with T0 |
| pH | RT | 4.61 | 4.52 |
| Viscosity (Brookfield | RT | 14697 | 12477 |
| LVDVII+) in cps | | | |
| S63/6 RPM | | | |
| Haake rheology | RT | 35/87/175 | 33/84/175 |
| (4 s⁻¹/20 s⁻¹/100 s⁻¹) | | | |

### Chemical Stability

Adapalene:

| Stability | | | |
|---|---|---|---|
| conditions | T0 | T + 1 month | T + 2 months |
| RT | 99.2% LC | 100.8% LC | NA |
| 40° C. | NA | 101.2% LC | NA |

Benzoyl Peroxide:

| Stability | | | |
|---|---|---|---|
| conditions | T0 | T + 1 month | T + 2 months |
| RT | 103.3% LC | 103% LC | NA |
| 40° C. | NA | 100.4% LC | 97% LC |

### EXAMPLE 9

### Formulation of Suspension Type Containing 0.3% Adapalene and 5.0% Benzoyl Peroxide

| Constituents | Concentration (%) |
|---|---|
| Adapalene | 0.30 |
| BPO | 5.00 |
| Sodium acryloyldimethyltaurate | 2.00 |
| copolymer/isohexadecane/polysorbate 80 | |
| Microcrystalline cellulose and | 1.50 |
| sodium carboxymethyl cellulose | |
| Sucrose laurate | 3.00 |
| Glycerol | 4.00 |
| Sodium docusate USP | 0.05 |
| Ethylenediaminetetraacetic acid | 0.05 |
| Propylene glycol | 4.00 |
| Poloxamer 124 | 0.20 |
| Purified water | qs 100% |

Specifications at T0 of the formulation base of suspension type:

Characterization at T0

| | |
|---|---|
| Macroscopic appearance | Slightly aerated opaque |
| | white suspension, flows at T0 |
| Microscopic appearance | Homogeneous dispersion of |
| | the active agents |
| pH | 5.31 |
| Viscosity (Brookfield | 21545 |
| LVDVII+) in | |
| cps S64/12 RPM | |
| Haake rheology | 27/40/64 |
| (4 s⁻¹/20 s⁻¹/100 s⁻¹) | |

### Physical Stability of the Formulation Base:

| | | T + | T + | T + |
|---|---|---|---|---|
| | | 1 month | 2 months | 3 months |
| Macroscopic | RT | In accor- | In accor- | In accor- |
| appearance | | dance | dance | dance |
| | | with T0 | with T0 | with T0 |
| | 40° C. | In accor- | In accor- | In accor- |
| | | dance | dance | dance |
| | | with T0 | with T0 | with T0 |
| Microscopic | RT | In accor- | In accor- | In accor- |
| appearance | | dance | dance | dance |
| | | with T0 | with T0 | with T0 |
| pH | RT | 5.16 | 5.10 | 4.94 |
| Viscosity | RT | 18046 | 19696 | 15847 |
| (Brookfield | | | | |
| LVDVII+) in cps | | | | |
| S64/12 RPM | | | | |
| Haake rheology | RT | 30/46/65 | 27/46/62 | 28/48/64 |
| (4 s⁻¹/20 | | | | |
| s⁻¹/100 s⁻¹) | | | | |

### Chemical Stability

Adapalene:

| Stability | | T + 1 | T + 2 | T + 3 |
|---|---|---|---|---|
| conditions | T0 | month | months | months |
| RT | 99.3% LC | 98.9% LC | NA | 98.9% LC |
| 40° C. | NA | 97.3% LC | NA | 97.8% LC |

Benzoyl Peroxide:

| Stability | | T + 1 | T + 2 | T + 3 |
|---|---|---|---|---|
| conditions | T0 | month | months | months |
| RT | 103.1% LC | 103.1% LC | NA | 100.9% LC |
| 40° C. | NA | 101.7% LC | 99.3% LC | 97.6% LC |

### EXAMPLE 10

### Formulation of Suspension Type Containing 0.1% Adapalene and 2.5% Benzoyl Peroxide

| Constituents | Concentration (%) |
|---|---|
| Adapalene | 0.10 |
| BPO | 2.50 |
| Sodium acryloyldimethyltaurate | 2.00 |
| copolymer/isohexadecane/polysorbate 80 | |
| Sclerotium rolfsii | 0.70 |
| Sucrose laurate | 3.00 |
| Glycerol | 4.00 |
| Sodium docusate | 0.05 |
| Ethylenediaminetetraacetic acid | 0.10 |
| Propylene glycol | 4.00 |
| Poloxamer 124 | 0.20 |
| Purified water | qs 100% |

Specifications at T0 of the formulation base of suspension type:

Characterization at T0

| | |
|---|---|
| Macroscopic appearance | Slightly aerated white |
| | suspension |
| Microscopic appearance | Homogeneous dispersion of |
| | the active agents |
| pH | 4.94 |
| Viscosity (Brookfield | 20775 |
| RVDVII+) in | |
| cps S27/10 RPM | |
| Haake rheology | 65/85/154 |
| (4 s⁻¹/20 s⁻¹/100 s⁻¹) | |

### Physical Stability of the Formulation Base:

| | | T + | T + | T + |
|---|---|---|---|---|
| | | 1 month | 2 months | 3 months |
| Macroscopic | RT | In accor- | In accor- | In accor- |
| appearance | | dance | dance | dance |
| | | with T0 | with T0 | with T0 |
| | 40° C. | In accor- | In accor- | In accor- |
| | | dance | dance | dance |
| | | with T0 | with T0 | with T0 |
| Microscopic | RT | In accor- | In accor- | In accor- |
| appearance | | dance | dance | dance |
| | | with T0 | with T0 | with T0 |
| pH | RT | 4.81 | 4.65 | 4.60 |
| Viscosity | RT | 18800 | 18225 | 21800 |
| (Brookfield | | | | |
| RVDVII+) in | | | | |
| cps S27/10 RPM | | | | |
| Haake rheology | RT | 62/87/137 | 66/97/147 | NA |
| (4 s⁻¹/20 | | | | |
| s⁻¹/100 s⁻¹) | | | | |

### Chemical Stability

Adapalene:

| Stability | | T + 1 | T + 2 | T + 3 |
|---|---|---|---|---|
| conditions | T0 | month | months | months |
| RT | 97.2% LC | 98.9% LC | NA | 97.7% LC |
| 40° C. | NA | 99.7% LC | 98.8% LC | 97.7% LC |

Benzoyl Peroxide:

| Stability | | T + 1 | T + 2 | T + 3 |
|---|---|---|---|---|
| conditions | T0 | month | months | months |
| RT | 100.9% LC | 100.1% LC | NA | 100% LC |
| 40° C. | NA | 102.6% LC | 96% LC | 93.4% LC |

### EXAMPLE 11

### Formulation of Suspension Type Containing 0.1% Adapalene and 2.5% Benzoyl Peroxide

| Constituents | Concentration (%) |
|---|---|
| Adapalene | 0.10 |
| BPO | 2.50 |
| Sclerotium rolfsii | 0.50 |
| Sodium acryloyldimethyltaurate | 2.00 |
| copolymer/isohexadecane/polysorbate 80 | |
| Sucrose laurate | 3.00 |
| Sodium docusate | 0.05 |
| Vegetable glycerol | 4.00 |
| Propylene glycol | 4.00 |
| Poloxamer 124 | 0.20 |
| Ethylenediaminetetraacetic acid | 0.10 |
| Purified water | qs 100% |

Specifications at T0 of the formulation base of suspension type:

Characterization at T0

| | |
|---|---|
| Macroscopic appearance | Slightly aerated white |
| | suspension |
| | Flows at T0 |
| Microscopic appearance | Homogeneous dispersion of |
| | the active agents |
| pH | 4.96 |
| Viscosity (Brookfield | 15575 |
| RVDVII+) in | |
| cps S27/10 RPM | |
| Haake rheology | 42/74/134 |
| (4 s⁻¹/20 s⁻¹/100 s⁻¹) | |

### Physical Stability of the Formulation Base:

| | | T + | T + | T + |
|---|---|---|---|---|
| | | 1 month | 2 months | 3 months |
| Macroscopic | RT | In accor- | In accor- | In accor- |
| appearance | | dance | dance | dance |
| | | with T0 | with T0 | with T0 |
| | 40° C. | In accor- | In accor- | In accor- |
| | | dance | dance | dance |
| | | with T0 | with T0 | with T0 |
| Microscopic | RT | In accor- | In accor- | In accor- |
| appearance | | dance | dance | dance |
| | | with T0 | with T0 | with T0 |
| pH | RT | 4.71 | 4.46 | 4.48 |
| Viscosity | RT | 14300 | 15300 | 13975 |
| (Brookfield | | | | |
| RVDVII+) in | | | | |
| cps S27/10 RPM | | | | |
| Haake rheology | RT | 47/71/120 | 45/73/131 | 47/75/118 |
| (4 s⁻¹/20 | | | | |
| s⁻¹/100 s⁻¹) | | | | |

### Chemical Stability

Adapalene:

| Stability | | T + 1 | T + 2 | T + 3 |
|---|---|---|---|---|
| conditions | T0 | month | months | months |
| RT | 99.1% LC | 98.7% LC | NA | 99.1% LC |
| 40° C. | NA | 100.3% LC | NA | 101.4% LC |

Benzoyl Peroxide:

| Stability | | T + 1 | T + 2 | T + 3 |
|---|---|---|---|---|
| conditions | T0 | month | months | months |
| RT | 104.0% LC | 104.3% LC | NA | 102.6% LC |
| 40° C. | NA | 102.2% LC | 100.4% LC | 98.4% LC |

Example 12: Examples of production of foam according to the invention
Given below are examples of foams obtained from the intermediate
compositions of gel and/or suspension type that are introduced into
an aerosol container containing at least one propellant gas under
pressure, as described below:

| | | | Foam 3 | Foam 4 | Foam 5 | |
|---|---|---|---|---|---|---|
| | Foam 1 | Foam 2 | Exam- | Exam- | Exam- | Foam 6 |
| Propellant | Exam- | Exam- | ple 5 | ple 11 | ple 10 | Exam- |
| gas or | ple 3 | ple 4 | suspen- | suspen- | suspen- | ple 7 |
| mixture (%) | gel | gel | sion | sion | sion | gel |
| Propane/ | 6 | 10 | | | | 6 |
| butane | | | | | | |
| Propane/ | | | 6 | 8 | | |
| isobutane | | | | | | |
| Propane/ | | | | | 6 | |
| butane/ | | | | | | |
| isobutane | | | | | | |

1. A composition comprising a combination of adapalene and benzoyl peroxide wherein the composition is in the form of a foam and is obtained from an intermediate composition in the form of a gel or a suspension.

2. The composition as defined by claim 1, wherein the intermediate composition has a viscosity of greater than 8,000 cps.

3. The composition as defined by claim 1, wherein the composition has a viscosity of between 8000 8,000 cps and 32 000 32,000 cps.

4. The composition as defined by claim 1, wherein the composition comprises:
between 60% and 98% by weight, relative to the total weight of the composition, of a gel or suspension, and
between 2% and 40% by weight, relative to the total weight of the composition, of at least one propellant gas.

5. An intermediate composition in gel form, the intermediate composition comprising in a physiologically acceptable medium:
adapalene and benzoyl peroxide,
water,
at least one gelling agent and/or pH-independent gelling agent,
at least one surfactant,
at least one wetting agent,
optionally, a chelating agent,
optionally, at least one humectant and/or emollient,
optionally, one or more additives.

6. An intermediate composition in suspension form, the intermediate composition comprising in a physiologically acceptable medium:
adapalene and benzoyl peroxide,
water,
at least one gelling agent and/or pH-independent gelling agent,
optionally, one or more suspension agents,
at least one surfactant,
at least one wetting agent,
optionally, a chelating agent,
optionally, at least one humectant and/or emollient,
optionally, one or more additives.

7. The intermediate composition as defined by claim 5, wherein the composition comprises, as a weight percentage relative to the total weight of the composition, from 0.1% to 10% of at least one gelling agent and/or pH-independent gelling agent for the aqueous phase.

8. The intermediate composition as defined by claim 6, wherein the adapalene and benzoyl peroxide are in dispersed form in the composition.

9. A method of treating a keratinization disorder, the method comprising administering to a subject in need thereof a composition as defined by claim 1.

10. The intermediate composition in gel or suspension form as defined by claim 5, comprising on a weight basis relative to the total weight of the gel or suspension:
(a) 0.01% to 5% of adapalene and 2% to 7% of benzoyl peroxide in combination;
(b) 65% to 85% of water;
(c) 0.2% to 5% of at least one gelling agent and/or pH-independent gelling agent for the aqueous phase;
(d) 0% to 5% of at least one suspension agent and/or viscosity enhancer;
(e) 0.5% to 10% of at least one surfactant;
(f) 0.1% to 8% of at least one wetting agent;
(g) 0% to 1% of a chelating agent;
(h) 0% to 15% of at least one humectant and/or emollient; and
(i) 0% to 15% of one or more additives.

11. The intermediate composition as defined by claim 10, wherein the content of surfactant (e) is between 2% and 10% by weight relative to the total weight of the composition.

12. A process for obtaining the intermediate composition as defined by claim 6, the process comprising combining, in a physiologically acceptable medium: adapalene and benzoyl peroxide; water; gelling agent and/or pH-independent gelling agent; suspension agents; surfactant; wetting agent; chelating agent; humectant and/or emollient, and optionally, one or more additives.

13. The composition as defined by claim 4, wherein the gel or suspension is present in an amount from 80% to 96% by weight, relative to the total weight of the composition.

14. The composition as defined by claim 4, wherein the at least one propellant is present in an amount from 4% to 20% by weight, relative to the total weight of the composition.

15. The intermediate composition as defined by claim 6, wherein the composition comprises, as a weight percentage relative to the total weight of the composition, from 0.1% to 10% of at least one gelling agent and/or pH-independent gelling agent for the aqueous phase.

16. The intermediate composition as defined by claim 13, wherein the at least one gelling agent and/or pH-independent gelling agent for the aqueous phase is present in an amount from 0.2% to 5%, as a weight percentage relative to the total weight of the composition.

17. The intermediate composition as defined by claim 7, wherein the at least one gelling agent and/or pH-independent gelling agent is present in an amount of from 0.2% to 5%, as a weight percentage relative to the total weight of the composition.

18. the method as defined by claim 9, wherein the keratinization disorder is acne.
